(19) United States
(12) Patent Application Publication
(10) Pub. No.: US 2024/0148430 A1
(43) Pub. Date: May 9, 2024

(54) ELECTRODE FOR A HAND-HELD ELECTROSURGICAL INSTRUMENT AND METHOD OF MANUFACTURING AN ELECTRODE

(71) Applicant: OLYMPUS WINTER & IBE GMBH, Hamburg (DE)

(72) Inventors: Christoph KNOPF, Stockelsdorf (DE); Christian BROCKMANN, Hollenstedt (DE); Jiri LAVICKA, Prerov (CZ); Petr KOMINEK, Prerov (CZ)

(73) Assignee: OLYMPUS WINTER & IBE GMBH, Hamburg (DE)

(21) Appl. No.: 18/233,987

(22) Filed: Aug. 15, 2023

Related U.S. Application Data
(60) Provisional application No. 63/423,537, filed on Nov. 8, 2022.

(51) Int. Cl. A61B 18/14

(52) U.S. Cl. CPC A61B 18/14; A61B 2018/00077; A61B 2018/1405

(57) ABSTRACT

A method for producing an electrode and an electrode. An electrode for an electrosurgical handheld instrument includes an electrically conductive wire made of a plurality of portions. In this case, two portions of the wire R1 and L1, which directly adjoin two ends of the wire, are aligned both parallel to one another and straight. Moreover, the two first portions R1 and L1 are adjoined by two second portions R2 and L2, which are likewise aligned both parallel to one another and straight. In this case, the portion R2 directly adjoins the portion R1 and the portion L2 directly adjoins the portion L1. The two portions R2 and L2 in turn are connected to one another via a further portion.

The invention relates to an electrode for an electrosurgical handheld instrument in accordance with the preamble of claim 1. The invention also relates to a method for producing an electrode in accordance with the preamble of claim 9.

Generic electrosurgical handheld devices, in particular resectoscopes, are used predominantly in urology for electrosurgical work. In this context, these devices are usually used for resection and evaporation of tissue, for example tissue in the lower urinary tract. To this end, the handheld device, in particular the resectoscope, may comprise a longitudinally displaceable electrode carrier, a distal work end of which, following the insertion of the device into the body to be treated, can be advanced from a distal end of the instrument shaft of the handheld device. An electrosurgical electrode is arranged on the electrode carrier at a distal end. By way of example, this electrode may be in the form of a loop and, depending on the structure of the instrument, is pushed or pressed through the tissue for the purpose of manipulating the said tissue.

Radiofrequency electric current is applied to the electrode for the aforementioned application. In this case, the electrode should be prevented from making electrical contact with the shaft tube of the handheld device. In the case of such an electrical contact, a short circuit could cause a device defect or could lead to an unpredictable traumatization in the body to be treated. To avoid such short circuits, the handheld devices comprise an electrically insulating insulation insert, also referred to as insulation tip, at their distal end regions. In this case, the insulation insert may be fastened either to an inner shaft or shaft tube, in which an electrode carrier is guided, or to the outer shaft of the instrument. Since such handheld devices may also be designed for multiple use and should accordingly be sterilized or autoclaved regularly, the insulation insert is designed to be detachable for cleaning purposes.

The size of the instrument or its cross section is sought to be as small as possible in the case of the handheld instrument for minimally invasive treatment of patients described herein, so that there is as little traumatization of the patient during the treatment as possible. Similarly, undertaking the intervention is sought to be particularly efficient. The choice of electrode is of decisive importance for an efficient intervention. The optimal treatment goal can only be achieved with the correct, application-specific electrode. Especially the effective cross section of the electrode or work instrument relative to the cross section of the instrument may be of decisive importance. However, the effective cross section or size of the electrode is restricted by the shape and diameter of the shaft of the electrosurgical handheld instrument. Thus, it is not feasible for the effective cross section of the electrode to be greater than the cross section of the outer circumference of the shaft. However, the space available for the electrode is not exploited optimally in the known instruments. Approaches for a better exploitation of the available space pursue highly complicated electrode geometries, which firstly are very complex and hence expensive in terms of production and secondly require much outlay in quality control.

The invention is therefore based on the problem of creating a method for producing an electrode and an electrode which can be used particularly efficiently and which is producible particularly cost-effectively.

A solution to this problem is described by claim 1. Accordingly, provision is made for an electrode for an electrosurgical handheld instrument to consist of an electrically conductive wire made of a plurality of portions. In this case, two portions of the wire R1 and L1, which directly adjoin two ends of the wire, are aligned both parallel to one another and straight. Moreover, the two first portions R1 and L1 are adjoined by two second portions R2 and L2, which are likewise aligned both parallel to one another and straight. In this case, the portion R2 directly adjoins the portion R1 and the portion L2 directly adjoins the portion L1. The two portions R2 and L2 in turn are connected to one another via a further portion C. The production of the electrode is particularly simple as a result of the continuous wire having a mirror symmetric structure and shape changes always occurring in only one spatial dimension. Both production and quality control are particularly simple as a result of the electrode not having shapes with directional changes in more than one dimension from one portion to a subsequent portion. In this context, quality control is simplified inasmuch as bends in only one spatial direction can be tested particularly easily in automated or partly automated fashion, for example by using camera systems or profile projectors. As a result of this simplification of the structure of the electrode according to the invention, the production in particular can be designed particularly cost-effectively.

Preferably, provision is made for the lengths of the portions R2 and L2 to be 0.7 mm to 1.7 mm. The wire thicknesses of the portions R1 and L1 and/or R2 and L2 and preferably also of further portions can be 0.2 mm to 1.0 mm. It was found that this dimensioning is particularly advantageous for an efficient treatment of the patient and for a particularly cost-effective production of the electrode.

Preferably, the invention further provides for the two second portions R2 and L2 to include an angle α of 35° to 120°, preferably 70°, 45° or 110°, with the first portions R1 and L1 Different angles may be chosen between the two pairs of portions, depending on the type of application for the electrode. The portions R1 and R2 are located in a respective plane and the portions L1 and L2 are likewise, with these planes being aligned parallel to one another. As a result of this shaping of the various portions according to the invention, it is possible to produce a multiplicity of different electrode shapes in particularly simple and hence cost-effective fashion. Moreover, a further exemplary embodiment of the invention may provide for the bend radii between the portions R1 and R2 and likewise between L1 and L2 to be 0.1 mm to 1 mm.

In particular, provision is made according to the invention for a portion C between the portions R2 and L2 to have a radius of 2.0 mm to 3.6 mm, preferably 2.8 mm. This loop-like shape of the electrode can be put to particularly versatile use and moreover has great stability. Moreover, it is conceivable for the portion C to be straight and aligned at right angles to the portions R2 and L2. Alternatively, it is likewise conceivable for the portion C to have a V-shape or a trapezoidal form. The portion C substantially represents the portion of the electrode in which the tissue is manipulated. As a result of the corresponding movement of the electrode carrier, the portion C of the electrode is pulled or pressed through the tissue to be manipulated.

A further advantageous exemplary embodiment of the invention may provide for the portions R2, L2 and C to be located in one plane. Depending on the application of the electrode, it may be advantageous for the aforementioned portions to assume different angles. As a result of the segment-type shape described herein, which has been bent into a plurality of portions, it is easily possible to produce different shapes of an electrode using simple means.

By varying the aforementioned angles and lengths, it is possible to produce a multiplicity of different shapes and sizes, to be precise without the production method having to be adapted to this end.

It is likewise preferably conceivable for the portion C to form a roller electrode by virtue of a hollow cylinder being plugged on a straight portion C. In this case, the hollow cylinder may have a cylinder shape or barrel shape.

A method for solving the specified problem is described by the measures of claim 9. Accordingly, provision is made for a plurality of steps to be carried out in succession for the purpose of producing an electrode for an electrosurgical handheld instrument according to claim 1. In a first step, a straight wire is initially deformed in such a way that a central portion C is formed, with two free wire ends of the wire being bent towards one another for this purpose, with the result that the wire ends are positioned parallel to one another. Subsequently, the two wire ends are bent equally and in parallel relative to the portion C, with the remaining open wire ends forming the portions R1 and L1 (step 2). The production of the electrode can be generalized or standardized as a result of these deformation steps. Slight variation of the aforementioned steps allows production of a multiplicity of different electrodes, which may have different embodiments depending on the field of application. As a result of the simple one-dimensional deformation steps for the aforementioned portions, it is possible to dispense with the use of complicated tools. The method is particularly cost-effective as a result of this simplification in production.

In particular, provision can be made for the two wire ends to be bent through an angle of 35° to 120°, preferably 45°, 90° or 110° relative to the portion C to form the portions L1 and R1. As a result of the choice of these angles, it is possible to realize all applications of the electrode. Moreover, it is possible to optimize the electrode for other, possibly novel applications.

Preferably, provision can be made in step 2 for the two wire ends to be bent vis-à-vis the portion C in such a way that two further parallel and straight portions L2 and R2, which are located in the same plane as the portions L1 and R1, are formed between the portion C and the two parallel and straight portions L1 and R1. Accordingly, the portions R2 and L2 can be located in the same plane as the portion C, and the portions L1 and R1 can be located in the same plane as the portions R2 and L2. The five portions overall are consequently located in only two planes, as a result of which a particularly simple production method in two steps is rendered possible. This is particularly advantageous both in terms of production costs and quality monitoring of the electrodes or for quality control.

The method according to the invention can provide for the portion C to be formed in application-specific fashion between the portions R2 and L2 in step 1. Accordingly, it is conceivable for the portion C to be formed round, half-round, elliptical, polygonal, straight or V-shaped. It is also conceivable for the portion C to be dominated by further components, for example a roller, a pin, a button or the like.

A preferred exemplary embodiment of the invention is described in detail below with reference to the drawing, in which:

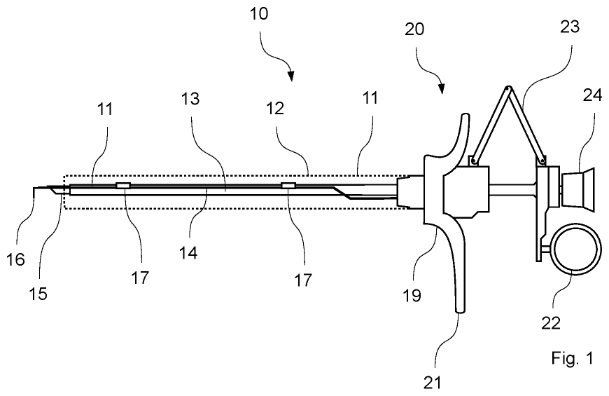

FIG. 1 shows a schematic illustration of a surgical handheld device, in particular a resectoscope,

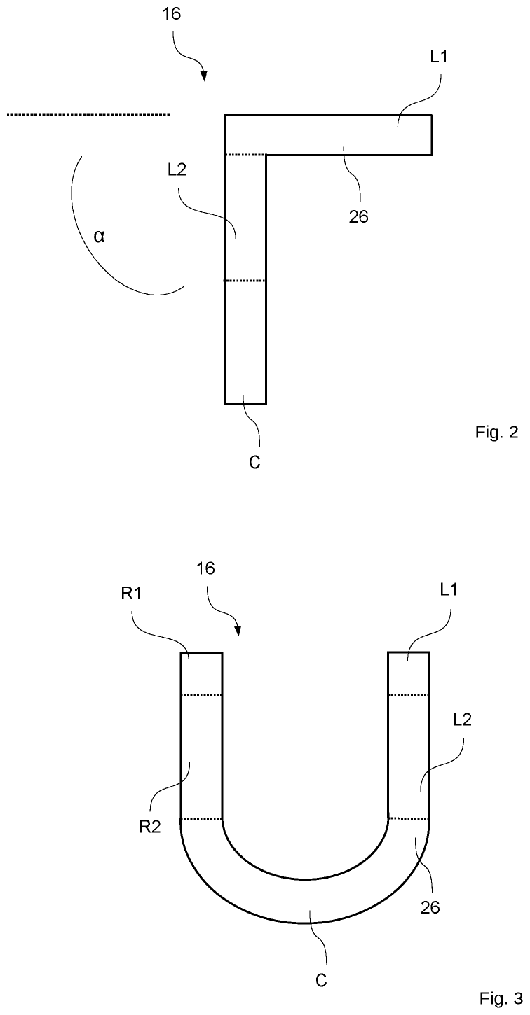

FIG. 2 shows a side view of an electrode,

FIG. 3 shows a front view of the electrode according to FIG. 2,

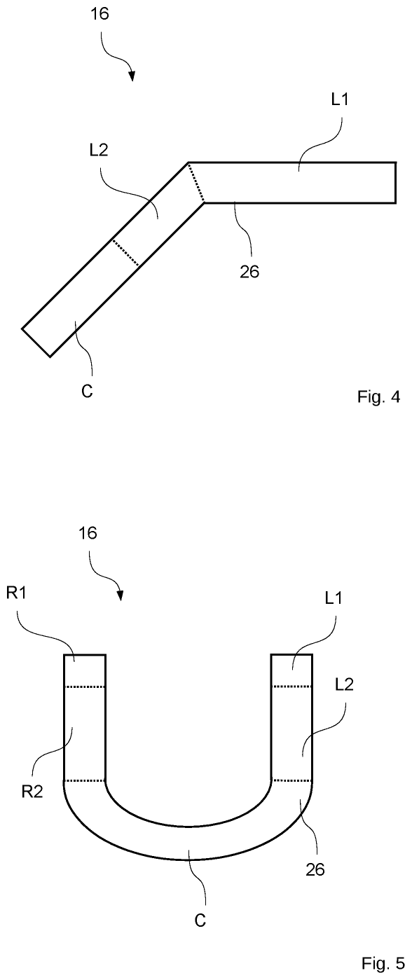

FIG. 4 shows a side view of a further exemplary embodiment of an electrode,

FIG. 5 shows a front view of the electrode according to FIG. 4,

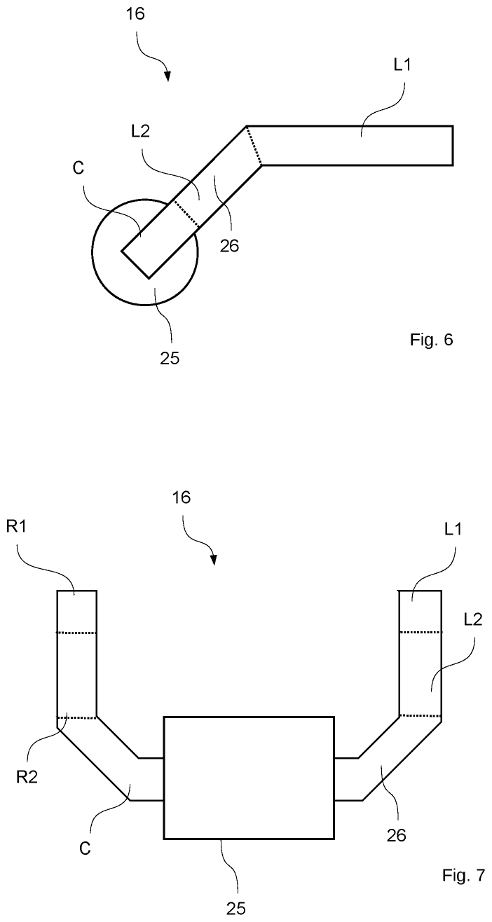

FIG. 6 shows a side view of a further exemplary embodiment of an electrode,

FIG. 7 shows a front view of the electrode according to FIG. 6,

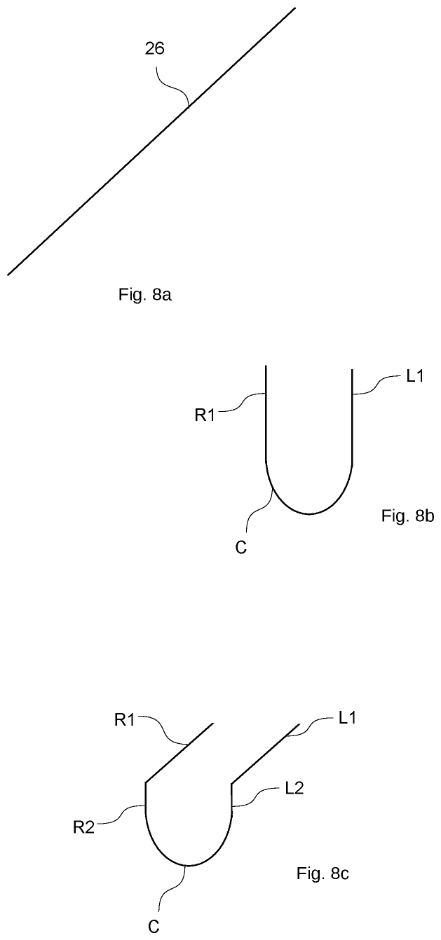

FIG. 8a shows a representation of a wire,

FIG. 8b shows step 1 of a production method for an electrode, and

FIG. 8c shows step 2 of the production method for the electrode.

FIG. 1 shows a schematic, lateral sectional illustration of a known resectoscope 10. The resectoscope 10 has a resectoscope shaft 11, which comprises an illustrated outer shaft 12 or enveloping tube. A tube-like inner shaft 13 extends within the outer shaft 12. An electrode carrier 14 and an indicated optical unit 15 are illustrated within the inner shaft 13, Moreover, further elements (not illustrated here) may be arranged within the resectoscope 10, for example a separate rinsing tube and the like.

The electrode carrier 14 has an electrosurgical tool or electrode 16 at a distal end. The electrode 16 illustrated here is represented as a loop, but it may also be formed as a button or the like.

The electrode carrier 14 can be moved axially in the distal and proximal direction in positively guided fashion by the actuation of a handle 19. In the process, it may be pushed beyond the distal end of the inner shaft 13 and outer shaft 12. This allows the surgeon to also manipulate tissue removed further away from the resectoscope tip. For this purpose, the inner shaft 13 and/or the electrode carrier 14 may further be mounted rotatably about their longitudinal axis. Radiofrequency electric current is applied to the electrode 16 for the manipulation of the tissue.

The resectoscope 10 illustrated in FIG. 1 has a passive transporter, in which a carriage 20 is displaced in the distal direction against the distal, first grip part 21 counter to a spring force applied by a spring bridge 23 as a result of relative movement between the grip parts 21 and 22 arranged proximally at the resectoscope shaft 11. When the carriage 20 is displaced in the distal direction against the grip part 21, the electrode carrier 14 is displaced in the distal direction (in a manner not illustrated). When pressure is removed from the handle parts 21, 22, the spring force produced by the spring bridge 23 forces the carriage 20 back into its initial position, with the electrode carrier 14 being pulled in the proximal direction. When the carriage 20 is displaced backwards, an electrosurgical intervention can be carried out without a manual force applied by the surgeon, which is to say passively, using the electrode 16.

For the targeted treatment by means of the electrode 16, the optical unit 15 is positioned in such a way that the surgeon has an optimal view of the operation region. To this end, the resectoscope 10 has at a proximal end an eyepiece 24 which is connected to the optical unit 15. Alternatively, it is also conceivable that a camera is arranged at the resectoscope 10 instead of the eyepiece 24.

The following figures depict a plurality of exemplary embodiments of electrodes 16, which all follow the same structure. Thus, these electrodes each have first portions R1 and L1 with the first portions R1 and L1 having the same length and being aligned parallel to one another. These first portions R1 and L1 are coupled to the distal ends of the electrode carrier tubes. This coupling to the electrode carrier tubes stabilizes the electrode 16 and applies electrical power thereto. The two second portions R2 and L2 adjoin the first portions R1 and L1. These two second portions R2 and L2 likewise have the same length and are aligned parallel to one another. The transition from the first portions R1 and L1 to the second portions R2 and L2 is implemented within a plane, which is to say the production process is particularly simple and does not require complicated tools (see FIGS. 2 to 7).

In the exemplary embodiment illustrated in FIGS. 2 to 5, the second portions R2 and L2 are inclined vis-à-vis the first portions R1 and L1 through an angle of 90° or 45°. Provision is made for this angle α to adopt a value between 35° and 120°, preferably 45°, 90° or 110°, or any other angle.

The portion C is located between the two second portions R2 and L2. This portion C connects the two second portions R2 and L2 and is in the form of a loop in the exemplary embodiments illustrated in FIGS. 2 to 5. The shape of the portion C may likewise vary and for example have a larger or smaller radius of curvature. In the present exemplary embodiment, the portion C is located in the same plane as the portions R2 and L2. To carry out the operation, the electrode 16 is pulled or pushed through the tissue to be treated with the portion C, depending on the type of resectoscope 10. To this end, provision is preferably made for the wire 26 of the electrode 16 to have a circular cross section.

FIGS. 6 and 7 depict what is known as a roller electrode 16. Just like the previous electrodes 16, this electrode 16 is also constructed from two first portions R1 and L1 and two second portions R2 and L2, which adjoin the first portions R1 and L1 and are at an angle vis-à-vis the first portions R1 and L1. A trapezoidal portion C, on which a roller 25 is situated, is arranged between the two second portions R2 and L2. The tissue to be manipulated is treated with this roller 25 during the treatment.

Even more shapes of the electrode 16 are conceivable in addition to the exemplary embodiments illustrated here. However, what is decisive is that all electrodes 16 are constructed from a wire 26 from which the aforementioned portions are formed, the said portions always having only one directional change in one plane among themselves. This prevents the electrode 16 from adopting a complicated geometry which is particularly difficult to produce and particularly difficult to control in terms of its quality. It is possible to standardize production as a result of the structure made of three different portions described here, leading to a very cost-effective production method.

FIGS. 8a to 8c very schematically sketch out the production method according to the invention for producing the electrode 16. Accordingly, a straight wire 26 (FIG. 8a), which may already have the overall length of the electrode 16 or else may be longer, is initially bent centrally in a first step so that the two portions R1 and L1 are formed to have the same length and be aligned parallel to one another (FIG. 8b). In the process, the portion C, which connects the two portions R1 and L1 to one another, may be bent about a shape which then determines the design of the portion C. This first shaping step of the wire 26 is consequently implemented in one plane. The second processing step for the wire 26 is now implemented in a plane aligned transversely to the plane determined by the portion C. As shown in FIG. 8c, the portions R1 and L1 are bent equally in a direction transversely to portion C in a second step, with the portions R1 and L1 maintaining their parallel alignment. In the exemplary embodiment of the method shown in FIG. 8c, bending is carried out in such a way that straight parallel portions remain between the portion C and the portions R1 and L1 These portions form the second portions R2 and L2. The shape or effective cross section of the electrode can be varied by varying the length of these portions R2 and L2 and also by varying the shape of the portion C. If required, the length of the portions R1 and L1 can still be adapted following the completion of the production method, in order to meet the dimensions of the electrosurgical handheld instrument. It is equally conceivable that the free ends of the portions R1 and L1 are provided with further contact means in order to improve the electrical contact with the electrode carrier tubes.

Consequently, it is substantially only necessary to carry out two bending steps in two planes in order to produce the electrode 16. As a result, it is not only the production of the electrode 16 which is particularly simple, but also the testing of the quality of the electrode 16.

### LIST OF REFERENCE SIGNS

10 Resectoscope
11 Resectoscope shaft
12 Outer shaft
13 Inner shaft
14 Electrode carrier
15 Optical unit
16 Electrode
17 Guiding element
18 Longitudinal axis
19 Handle
20 Carriage
21 Grip part
22 Grip part
23 Spring bridge
24 Eyepiece
25 Roller
26 Wire
R1 First portion
R2 Second portion
L1 First portion
L2 Second portion
C Central portion
α Angle

1. An electrode for an electrosurgical handheld instrument consisting of an electrically conductive wire, with the two ends of the wire being couplable to an electrode carrier of the handheld instrument, wherein two portions R1 and L1 of the wire, which adjoin the two ends of the wire, are aligned both parallel to one another and straight, with two second portions R2 and L2, likewise aligned both parallel to one another and straight, adjoining the first portions R1 and L1.

2. The electrode for an electrosurgical handheld instrument according to claim 1, wherein the lengths of the portions R2 and L2 are 0.7 mm to 1.7 mm.

3. The electrode for an electrosurgical handheld instrument according to claim 1, wherein the wire thicknesses of the portions R1 and L1 and/or R2 and L2 are 0.2 mm to 1.0 mm.

4. The electrode for an electrosurgical handheld instrument according to claim 1, wherein the two second portions R2 and L2 include an angle α of 35° to 120° with the first portions R1 and L1.

5. The electrode for an electrosurgical handheld instrument according to claim 1, wherein the bend radii between the portions R1 and R2 and likewise between L1 and L2 are 0.1 mm to 1 mm.

6. The electrode for an electrosurgical handheld instrument according to claim 1, wherein a portion C, which has a radius of 2.0 mm to 3.6 mm, or which is straight extends between the two portions R2 and L2, and the portion C is aligned at a right angle to the portions R2 and L2 or has a V-shape or a trapezoidal form.

7. The electrode for an electrosurgical handheld instrument according to claim 6, wherein the portions R2, L2 and C are located in a plane.

8. The electrode for an electrosurgical handheld instrument according to claim 6, wherein the portion C forms a roller electrode by virtue of a roller being plugged on a straight portion C.

9. A method for producing an electrode for an electrosurgical handheld instrument according to claim 1, the electrode consisting of an electrically conductive wire and the two ends of the wire being couplable to an electrode carrier of the handheld instrument, wherein initially a straight wire is deformed in such a way that a central portion C is formed, with two free wire ends of the wire being bent towards one another for this purpose, with the result that the wire ends are positioned parallel to one another (step 1), and with the two wire ends subsequently being bent equally and in parallel relative to the portion C, with the remaining open wire ends forming the portions R1 and L1 (step 2).

10. The method for producing an electrode according to claim 9, wherein the two wire ends are bent through an angle of 35° to 120° relative to the portion C to form the portions L1 and R1.

11. The method for producing an electrode according to claim 9, wherein in step 2 the two wire ends are bent vis-à-vis the portion C in such a way that two further parallel and straight portions L2 and R2, which are located in the same plane as the portions L1 and R1, are formed between the portion C and the two parallel and straight portions L1 and R1.

12. The method for producing an electrode according to claim 9, wherein in step 1 the portion C is formed round, half-round, straight, V-shaped or the like in application-specific fashion.
